SEQUENCE LISTING (27 sequences)

SEQ ID NO: 1 - protein, 1093 aa, synthetic construct
  REGION 1..1093: note = Recombinant Human Slit2-N
QACPAQCSCSGSTVDCHGLALRSVPRNIPRNTERLDLNGNNITRITKTDFAGLRHLRVLQLMENKISTIERGAFQDLKELERLRLNRNHLQLFPELLFLGTAKLYRLDLSENQIQAIPRKAFRGAVDIKNLQLDYNQISCIEDGAFRALRDLEVLTLNNNNITRLSVASFNHMPKLRTFRLHSNNLYCDCHLAWLSDWLRQRPRVGLYTQCMGPSHLRGHNVAEVQKREFVCSGHQSFMAPSCSVLHCPAACTCSNNIVDCRGKGLTEIPTNLPETITEIRLEQNTIKVIPPGAFSPYKKLRRIDLSNNQISELAPDAFQGLRSLNSLVLYGNKITELPKSLFEGLFSLQLLLLNANKINCLRVDAFQDLHNLNLLSLYDNKLQTIAKGTFSPLRAIQTMHLAQNPFICDCHLKWLADYLHTNPIETSGARCTSPRRLANKRIGQIKSKKFRCSAKEQYFIPGTEDYRSKLSGDCFADLACPEKCRCEGTTVDCSNQKLNKIPEHIPQYTAELRLNNNEFTVLEATGIFKKLPQLRKINFSNNKITDIEEGAFEGASGVNEILLTSNRLENVQHKMFKGLESLKTLMLRSNRITCVGNDSFIGLSSVRLLSLYDNQITTVAPGAFDTLHSLSTLNLLANPFNCNCYLAWLGEWLRKKRIVTGNPRCQKPYFLKEIPIQDVAIQDFTCDDGNDDNSCSPLSRCPTECTCLDTVVRCSNKGLKVLPKGIPRDVTELYLDGNQFTLVPKELSNYKHLTLIDLSNNRISTLSNQSFSNMTQLLTLILSYNRLRCIPPRTFDGLKSLRLLSLHGNDISVVPEGAFNDLSALSHLAIGANPLYCDCNMQWLSDWVKSEYKEPGIARCAGPGEMADKLLLTTPSKKFTCQGPVDVNILAKCNPCLSNPCKNDGTCNSDPVDFYRCTCPYGFKGQDCDVPIHACISNPCKHGGTCHLKEGEEDGFWCICADGFEGENCEVNVDDCEDNDCENNSTCVDGINNYTCLCPPEYTGELCEEKLDFCAQDLNPCQHDSKCILTPKGFKCDCTPGYVGEHCDIDFDDCQDNKCKNGAHCTDAVNGYTCICPEGYSGLFCEFSPPMV

SEQ ID NO: 2 - DNA, 18 bp, synthetic construct
  misc_feature 1..18: note = IL8(CXCL8) primer - Forward
  source 1..18: mol_type = other DNA
ctggccgtggctctcttg

SEQ ID NO: 3 - DNA, 20 bp, synthetic construct
  misc_feature 1..20: note = IL8(CXCL8) primer - Reverse
  source 1..20: mol_type = other DNA
ccttggcaaaactgcacctt

SEQ ID NO: 4 - DNA, 20 bp, synthetic construct
  misc_feature 1..20: note = IL1b primer - Forward
  source 1..20: mol_type = other DNA
ctgtcctgcgtgttgaaaga

SEQ ID NO: 5 - DNA, 23 bp, synthetic construct
  misc_feature 1..23: note = IL1b primer - Reverse
  source 1..23: mol_type = other DNA
ttgggtaatttttgggatctaca

SEQ ID NO: 6 - DNA, 18 bp, synthetic construct
  misc_feature 1..18: note = CCL2 primer - Forward
  source 1..18: mol_type = other DNA
ctctgccgcccttctgtg

SEQ ID NO: 7 - DNA, 18 bp, synthetic construct
  misc_feature 1..18: note = CCL2 primer - Reverse
  source 1..18: mol_type = other DNA
tgcatctggctgagcgag

SEQ ID NO: 8 - DNA, 20 bp, synthetic construct
  misc_feature 1..20: note = CCL5 primer - Forward
  source 1..20: mol_type = other DNA
atgaaggtctccgcggcagc

SEQ ID NO: 9 - DNA, 20 bp, synthetic construct
  misc_feature 1..20: note = CCL5 primer - Reverse
  source 1..20: mol_type = other DNA
ctagctcatctccaaagagt

SEQ ID NO: 10 - DNA, 19 bp, synthetic construct
  misc_feature 1..19: note = CXCL1 primer - Forward
  source 1..19: mol_type = other DNA
cttgcctcaatcctgcatc

SEQ ID NO: 11 - DNA, 21 bp, synthetic construct
  misc_feature 1..21: note = CXCL1 primer - Reverse
  source 1..21: mol_type = other DNA
ccttctggtcagttggatttg

SEQ ID NO: 12 - DNA, 20 bp, synthetic construct
  misc_feature 1..20: note = CXCL2 primer - Forward
  source 1..20: mol_type = other DNA
tgcagggaattcacctcaag

SEQ ID NO: 13 - DNA, 20 bp, synthetic construct
  misc_feature 1..20: note = CXCL2 primer - Reverse
  source 1..20: mol_type = other DNA
tgagacaagctttctgccca

SEQ ID NO: 14 - DNA, 20 bp, synthetic construct
  misc_feature 1..20: note = SAA1 primer - Forward
  source 1..20: mol_type = other DNA
tttctgctccttggtcctgg

SEQ ID NO: 15 - DNA, 20 bp, synthetic construct
  misc_feature 1..20: note = SAA1 primer - Reverse
  source 1..20: mol_type = other DNA
ctctggcatcggtgatcact

SEQ ID NO: 16 - DNA, 19 bp, synthetic construct
  misc_feature 1..19: note = CXCL12 primer - Forward
  source 1..19: mol_type = other DNA
tgccagagccaacgtcaag

SEQ ID NO: 17 - DNA, 20 bp, synthetic construct
  misc_feature 1..20: note = CXCL12 primer - Reverse
  source 1..20: mol_type = other DNA
cagccgggctacaatctgaa

SEQ ID NO: 18 - DNA, 21 bp, synthetic construct
  misc_feature 1..21: note = IL6 primer - Forward
  source 1..21: mol_type = other DNA
aattcggtacatcctcgacgg

SEQ ID NO: 19 - DNA, 20 bp, synthetic construct
  misc_feature 1..20: note = IL6 primer - Reverse
  source 1..20: mol_type = other DNA
ggttgttttctgccagtgcc

SEQ ID NO: 20 - DNA, 24 bp, synthetic construct
  misc_feature 1..24: note = Slit2 primer - Forward
  source 1..24: mol_type = other DNA
gtcaatgacctactggcctcattc

SEQ ID NO: 21 - DNA, 25 bp, synthetic construct
  misc_feature 1..25: note = Slit2 primer - Reverse
  source 1..25: mol_type = other DNA
tgattgctacacaccatttcgtttc

SEQ ID NO: 22 - protein, 122 aa, synthetic construct
  REGION 1..122: note = SAA1 Antibody
MKLLTGLVFCSLVLGVSSRSFFSFLGEAFDGARDMWRAYSDMREANYIGSDKYFHARGNYDAAKRGPGGVWAAEAISDARENIQRFFGHGAEDSLADQAANEWGRSGKDPNHFRPAGLPEKY

SEQ ID NO: 23 - protein, 89 aa, synthetic construct
  REGION 1..89: note = Slit2 Antibody
LYINSELQDFQKVPMQTGILPGCEPCHKKVCAHGTCQPSSQAGFTCECQEGWMGPLCDQRTNDPCLGNKCVHGTCLPINAFSYSCKCLE

SEQ ID NO: 24 - protein, 414 aa, synthetic construct
  REGION 1..414: note = Slit2 Antibody
SPPMVLPRTSPCDNFDCQNGAQCIVRINEPICQCLPGYQGEKCEKLVSVNFINKESYLQIPSAKVRPQTNITLQIATDEDSGILLYKGDKDHIAVELYRGRVRASYDTGSHPASAIYSVETINDGNFHIVELLALDQSLSLSVDGGNPKIITNLSKQSTLNFDSPLYVGGMPGKSNVASLRQAPGQNGTSFHGCIRNLYINSELQDFQKVPMQTGILPGCEPCHKKVCAHGTCQPSSQAGFTCECQEGWMGPLCDQRTNDPCLGNKCVHGTCLPINAFSYSCKCLEGHGGVLCDEEEDLFNPCQAIKCKHGKCRLSGLGQPYCECSSGYTGDSDREISCRGERIRDYYQKQQGYAACQTTKKVSRLECRGGCAGGQCCGPLRSKRRKYSFECTDGSSFVDEVEKVVKCGCTRCV

SEQ ID NO: 25 - protein, 44 aa, synthetic construct
  REGION 1..44: note = p53 Antibody
MEEPQSDPSVEPPLSQETFSDLWKLLPENNVLSPLPSQAMDDLM

SEQ ID NO: 26 - protein, 164 aa, synthetic construct
  REGION 1..164: note = p21 Antibody
MSEPAGDVRQNPCGSKACRRLFGPVDSEQLSRDCDALMAGCIQEARERWNFDFVTETPLEGDFAWERVRGLGLPKLYLPTGPRRGRDELGGGRRPGTSPALLQGTAEEDHVDLSLSCTLVPRSGEQAEGSPGGPGDSQGRKRRQTSMTDFYHSKRRLIFSKRKP

SEQ ID NO: 27 - protein, 875 aa, synthetic construct
  REGION 1..875: note = recombinant mouse Slit2-N
QACPAQCSCSGSTVDCHGLALRSVPRNIPRNTERLDLNGNNITRITKTDFAGLRHLRVLQLMENRISTIERGAFQDLKELERLRLNRNNLQLFPELLFLGTAKLYRLDLSENQIQAIPRKAFRGAVDIKNLQLDYNQISCIEDGAFRALRDLEVLTLNNNNITRLSVASFNHMPKLRTFRLHSNNLYCDCHLAWLSDWLRQRPRVGLYTQCMGPSHLRGHNVAEVQKREFVCSGHQSFMAPSCSVLHCPAACTCSNNIVDCRGKGLTEIPTNLPETITEIRLEQNSIRVIPPGAFSPYKKLRRLDLSNNQISELAPDAFQGLRSLNSLVLYGNKITELPKSLFEGLFSLQLLLLNANKINCLRVDAFQDLHNLNLLSLYDNKLQTVAKGTFSALRAIQTMHLAQNPFICDCHLKWLADYLHTNPIETSGARCTSPRRLANKRIGQIKSKKFRCSGTEDYRSKLSGDCFADLACPEKCRCEGTTVDCSNQRLNKIPDHIPQYTAELRLNNNEFTVLEATGIFKKLPQLRKINFSNNKITDIEEGAFEGASGVNEILLTSNRLENVQHKMFKGLESLKTLMLRSNRISCVGNDSFIGLGSVRLLSLYDNQITTVAPGAFDSLHSLSTLNLLANPFNCNCHLAWLGEWLRRKRIVTGNPRCQKPYFLKEIPIQDVAIQDFTCDDGNDDNSCSPLSRCPSECTCLDTVVRCSNKGLKVLPKGIPKDVTELYLDGNQFTLVPKELSNYKHLTLIDLSNNRISTLSNQSFSNMTQLLTLILSYNRLRCIPPRTFDGLKSLRLLSLHGNDISVVPEGAFNDLSALSHLAIGANPLYCDCNMQWLSDWVKSEYKEPGIARCAGPGEMADKLLLTTPSKKFTCQ

(19) United States
(12) Patent Application Publication
(10) Pub. No.: US 2025/0288642 A1
(43) Pub. Date: Sep. 18, 2025

(54) COMPOSITION FOR INDUCING REVERSE-AGING CONTAINING SLIT2 AND METHOD FOR INDUCING REVERSE-AGING

(71) Applicant: AJOU UNIVERSITY INDUSTRY-ACADEMIC COOPERATION FOUNDATION, Suwon-si, Gyeonggi-do (KR)

(72) Inventors: Tae-Jun PARK, Seongnam-si (KR); Jang-Hee KIM, Suwon-si (KR); Young Hwa KIM, Suwon-si (KR); Young-Kyoung LEE, Suwon-si (KR)

(73) Assignee: AJOU UNIVERSITY INDUSTRY-ACADEMIC COOPERATION FOUNDATION, Suwon-si, Gyeonggi-do (KR)

(21) Appl. No.: 18/862,252

(22) PCT Filed: May 3, 2023
(86) PCT No.: PCT/KR2023/006011

§ 371 Date: Nov. 1, 2024

(30) Foreign Application Priority Data
May 3, 2022 (KR) 10-2022-0054669

(51) Int. Cl. A61K 38/17; A61K 8/64; A61P 43/00; A61Q 7/00; A61Q 19/02; A61Q 19/08

(52) U.S. Cl. CPC A61K 38/1709; A61K 8/64; A61P 43/00; A61Q 7/00; A61Q 19/02; A61Q 19/08

(57) ABSTRACT

A reverse-aging or anti-aging composition containing Slit2 proteins, a pharmaceutical composition containing same for the prevention or treatment of cellular senescence-related diseases, and/or cosmetic composition containing same for anti-aging are disclosed. Also, a method for inducing reverse-aging of senescent cells, including the step of treating senescent cells with Slit2 proteins is disclosed. The composition, by minimizing tumor formation and cancer progression which are the side effects of conventional reverse-aging induction methods, may be useful as a composition for reverse-aging or anti-aging and/or a method for inducing reverse-aging.

### TECHNICAL FIELD

The present invention relates to a reverse-aging or anti-aging composition containing Slit2 proteins, a pharmaceutical composition for preventing or treating cellular aging-related diseases containing Slit2 proteins, and/or an anti-aging cosmetic composition containing Slit2 proteins. The present invention relates to a method of reverse-aging senescent cells including treating the senescent cells with Slit2 proteins.

### BACKGROUND ART

Aging (senescence) is a phenomenon in which the body functions of organisms deteriorate over time. Aging is generally characterized by a decrease in the ability to cope with stress, loss of homeostasis, and increased risk of disease. Changes that occur in humans over time accumulate due to aging (Bowen, Richard L.; Atwood, Craig S. (2004). Gerontology) 50 (5): 265-90), and these changes include physical, psychological, and social changes. When aging progresses beyond a predetermined level, organisms die.

Cellular aging refers to a phenomenon in which normal cells are in a state of proliferation inhibition where they no longer divide due to loss of various aging-induced stresses, such as telomere, DNA damage response, active oxygen stress, and tumor-induced stress. Senescent cells secrete various inflammatory factors through changes in metabolic pathways, which are known to promote aging of surrounding normal cells and tissues, and ultimately induce aging-related diseases and individual aging.

For a long time, humans have regarded aging as inevitable and aging has caused fear and anxiety. However, as the mechanism of aging has recently been identified, the view that aging is a disease that can be overcome has spread in the modern scientific community, and the World Health Organization (WHO) also recognized aging as a disease by adding “Code MG2A: Old age” to the “International Classification of Diseases” in 2018. Accordingly, as the view that aging is a phenomenon which we cannot do anything about has changed to the view that aging is a disease that can be overcome, research on anti-aging measures has also become active.

In an approach of the anti-aging measures, a great deal of research has been conducted on reverse aging methods to turn aged cells back into young cells. A representative example of reverse aging research reported that partial reprogramming by short-term cyclic expression of Oct4, Sox2, Klf4, and c-Myc (OSKM) improves cellular and physiological characteristics of aging and in vivo expression of OSKM improves cellular and physiological features of aging and extends lifespan in a mouse model of premature aging, and similarly, in vivo expression of OSKM improves recovery from metabolic disease and muscle damage in old wild-type mice (Ocampo, A. et al. Cell. 2016, 15; 167(7):1719-1733.e12).

However, Oct4 is critical to the self-renewal of undifferentiated embryonic stem cells, SOX2 is a transcription factor essential for maintaining self-renewal or pluripotency of undifferentiated embryonic stem cells, Klf4 is a zinc finger protein that can act on both transcriptional activation or inhibition of genes related to cell proliferation, differentiation, and apoptosis, and c-Myc contributes to cancer formation. Therefore, the OSKM-based anti-aging method has fatal side effects that induce tumorigenesis and cancer progression.

Under this background, the present inventors have made great efforts to overcome the side effects of conventional anti-aging methods, such as tumor formation and cancer progression, and as a result, the present inventors found that senescent cells are reverse-aged when the senescent cells and young cells are co-cultured, and found that senescent cells are reversed-aged when the senescent cells are treated with Slit2, which is a protein highly expressed in the young cells, and identified that the Slit2 protein is an anti-aging inducer, thereby completing the present invention.

The information disclosed in this Background section is provided only for better understanding of the background of the present invention, and therefore it may not include information that forms the prior art that is already obvious to those skilled in the art.

### DISCLOSURE

Therefore, the present invention has been made in view of the above problems, and it is one object of the present invention to provide the use of Slit2 protein for reverse-aging or anti-aging.

It is another object of the present invention to provide the use of Slit2 protein for the prevention or treatment of diseases related to cellular aging.

In order to accomplish the objects, the present invention provides a reverse-aging or anti-aging composition containing Slit2 protein.

The present invention also provides a method of inducing reverse-aging or anti-aging of senescent cells, including treating senescent cells with Slit2 protein.

The present invention also provides the use of Slit2 protein for reverse-aging or anti-aging.

The present invention also provides the use of Slit2 protein for the preparation of a reverse-aging or anti-aging composition.

The present invention also provides a pharmaceutical composition for preventing or treating a cellular aging-related disease, containing Slit2 protein.

The present invention also provides a method for preventing or treating a cellular aging-related disease, including administering Slit2 protein to a subject.

The present invention also provides the use of Slit2 protein for preventing or treating a cellular aging-related disease.

The present invention also provides the use of Slit2 protein for the preparation of a pharmaceutical composition for preventing or treating a cellular aging-related disease.

The present invention also provides an anti-aging cosmetic composition containing Slit2 protein.

The present invention also provides the use of Slit2 protein for the preparation of an anti-aging cosmetic composition.

### DESCRIPTION OF DRAWINGS

FIG. 1A shows the morphology of young cells, mid-old cells, and old cells when normal fibroblasts were subcultured.

FIG. 1B is a graph showing the expression patterns of p53, p21 (CDKN1A), and p16^{INK4A} and the cell growth patterns of young cells, mid-old cells, and old cells.

FIG. 1C is a graph showing the number of cells positive for senescence-associated β-galactosidase (SA-β-Gal), a marker of senescence, in young cells, mid-old cells, and old cells.

FIG. 1D is a graph showing the expression patterns of inflammation-related genes (IL8, IL1b, CCL2, CCL5, CXCL1, CXCL2, SAA1, and IL6) in young cells, mid-old cells, and old cells.

FIG. 1E is a graph showing the expression patterns of CXCL12 in young cells, mid-old cells, and old cells.

FIG. 2 is a graph showing the expression patterns of SAA1 and Slit2 genes in young cells and mid-old cells in vitro.

FIG. 3 shows the expression patterns of genes (p16^{INK4A}, p21, IL1b, SAA1, CXCL12, and Slit2) in young cells, mid-old cells, and old cells.

FIG. 4A shows the expression of p16^{INK4A}, a senescence marker, in young and old tissues, and FIG. 4B shows the expression of SAA1 and Slit2 proteins in young and old tissues.

FIG. 5A shows the morphology of cells 30 days after co-culture of mid-old cells and mid-old cells, and co-culture of young cells and mid-old cells. FIG. 5B shows a graph showing cell growth 30 days after co-culture.

FIG. 6 shows a graph showing the expression pattern of the Slit2 gene in young cells, mid-old cells, and old cells using real-time PCR.

FIG. 7A is a graph showing the morphology and cell count observed on day 8 after treating mid-old cells with 100 ng/ml of recombinant human Slit2-N protein. FIG. 7B shows the expression of p53 and p21 observed by western blot after treating mid-old cells with 10 ng/mL to 40 ng/mL of recombinant human Slit2-N protein for 2 days. FIG. 7C is a graph showing comparison in cell proliferation between mid-old cells treated with 100 ng/mL of recombinant human Slit2-N protein and the control group.

FIG. 8 is a graph showing the morphology and cell count observed on day 8 after treating old cells with 100 ng/mL of recombinant human Slit2-N protein.

FIG. 9 shows the activity of 22 month-aged male and female mice administered Slit2. The activity of mice was determined by measuring the distance traveled in the cage for 1 minute. Each mouse was measured three times and the average was expressed as the distance moved by that mouse.

### BEST MODE

Unless defined otherwise, all technical and scientific terms used herein have the same meanings as appreciated by those skilled in the field to which the present invention pertains. In general, the nomenclature used herein is well-known in the art and is ordinarily used.

The term “aging” refers to a phenomenon in which physiological functions necessary for survival and reproduction deteriorate over time. As aging progresses, an individual, organ, tissue, or cell gradually undergoes senescence. Through senescence, the ability to cope with stress generally decreases, homeostasis cannot be maintained, and the risk of developing a disease increases. As the mechanisms of aging have recently been identified, the view that aging is a disease that can be overcome has spread in the modern scientific community, and research on anti-aging aging has also become active. As one of the anti-aging measures, reverse aging methods that attempt to turn senescent cells back into young cells has been actively researched.

As used herein, the term “reverse aging” refers to a phenomenon in which the progression of the aging is reversed. In certain embodiments, senescent cells can recover the characteristics of young cells through reverse aging. Examples of the characteristics of the young cells include, but are not limited to, a decrease in cell size, an increase in cell proliferation capacity, an increase in Slit2 protein expression, and a decrease in expression of inflammation-related genes.

As used herein, the term “anti-aging” refers to inhibition or delay of the progression of the aging. Anti-aging can prevent or alleviate the negative effects of the aging.

As used herein, the term “anti-aging” refers to reversing the progression of the aging, and it may be considered that a substance that exhibits an anti-aging effect, such as the Slit2 protein, may also exhibit an anti-aging effect that inhibits the progression of aging.

In certain embodiments, young cells may be anti-aged, old cells may be reverse-aged, and mid-old cells may be reverse-aged and/or anti-aged.

In one embodiment of the present invention, it was found that Slit2 protein, among substances secreted from young cells, induces reverse aging of senescent cells, preferably mid-old cells. It was found that, when senescent cells are treated with the Slit2 protein, the cell growth of the senescent cells increase and the expression of inflammation-related genes decrease, so that the progression of aging can be reversed or inhibited (anti-aged) and thus the Slit2 protein may be useful as a reverse aging or anti-aging composition, a pharmaceutical composition for preventing or treating cellular aging-related diseases, and/or an anti-aging cosmetic composition.

In one aspect, the present invention provides a reverse-aging or anti-aging composition containing Slit2 protein.

In another aspect, the present invention provides a method of inducing reverse-aging or anti-aging of senescent cells using Slit2 protein.

In another aspect, the present invention provides the use of Slit2 protein for reverse-aging or anti-aging.

In another aspect, the present invention provides the use of Slit2 protein for the preparation of a reverse-aging or anti-aging composition.

The composition according to the present invention may be used in any one of various forms, for example, a pharmaceutical composition, a health functional food composition, a cosmetic composition, a reagent composition, or a medium supplement composition, but is not limited thereto.

As used herein, the term “Slit2 protein (Slit homolog 2 protein)” refers to a protein encoded by the Slit2 gene in humans (Chedotal A (February 2008). Adv Exp Med Biol. Advances in Experimental Medicine and Biology. 621: 65-80). Slit2 protein is a secreted axon guidance molecule that binds to ROBO-1, -2, -3, and -4, laminin-1, DAN, gremlin, and glypican-1. Slit2 protein binds to ROBO receptors and triggers axon repulsion, thereby regulating axon guidance. Slit2 protein has also been shown to induce growth cone collapse, inhibit migration of oligodendrocyte precursor cells, and promote axon elongation, branch formation, and fasciculation. Slit2 protein is cleaved into N-terminal and C-terminal fragments, designated “Slit2-N” and “Slit2-C”, respectively. Slit2-N and Slit2-C proteins have been shown to have distinct activities. Slit2-N protein binds to ROBO-1, thereby repelling motor axonal migration, whereas Slit2-C protein binds to glypican-1, thereby promoting motor axonal migration (Nguyen Ba-Charvet, K. T. et al. (2001) J. Neurosci. 21:4281). Outside the nervous system, Slit2 proteins play roles in a wide range of biological processes, including cell adhesion and migration, tumor progression and metastasis, angiogenesis, lymphangiogenesis, HIV-1 replication, platelet function and thrombus formation, and stem cellular aging, which is also known as an anti-inflammatory protein (Zhao H, et al., J Immunol. 2014 Jan. 1; 192(1):385-93. doi: 10.4049/jimmunol.1302021. Epub 2013 Nov. 22. PMID: 24272999; PMCID: PMC3908786).

In the present invention, the Slit2 protein may be a Slit2-N protein or a Slit2-C protein, and preferably, a Slit2-N protein.

In the present invention, the Slit2 protein may include a sequence selected from the group consisting of NCBI reference sequences NP_004778.1, NP_001276064.1, NP_001276065.1, XP 005248268.1, XP_006714049.1, XP_011512211.1, XP_016864334.1, and XP_011512212.2, and preferably, NP_004778.1.

Accordingly, in the present invention, the Slit2-N protein may be a recombinant human Slit2-N containing a sequence represented by SEQ ID NO. 1.

In the present invention, the Slit2 protein is used to mean not only a natural protein but also a recombinant protein and a fragment thereof, as long as its activity is maintained.

In an embodiment of the present invention, a recombinant human Slit2-N protein containing a sequence represented by SEQ ID NO. 1 was used (purchased from PEPROTECH, catalog number 150-11, Source: HEK293 cells), but is not limited thereto.

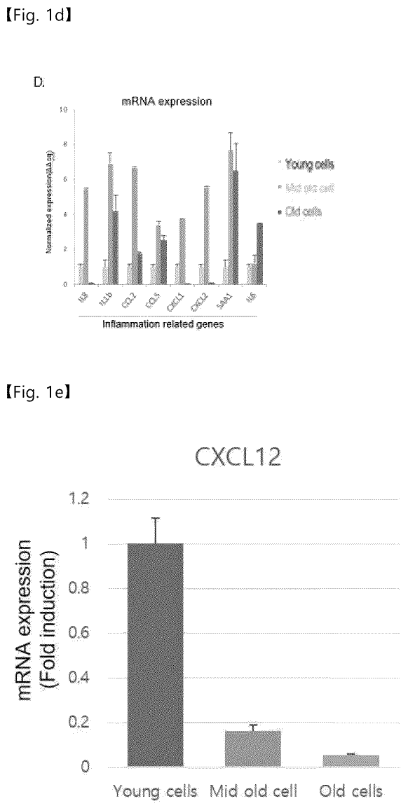

In one embodiment of the present invention, the characteristics of young cells, mid-old cells, and old cells were identified by passage culture, and in another embodiment of the present invention, the characteristics of mid-old cells, young cells, and old cells were identified in vitro. The characteristics are summarized in FIG. 3.

In another embodiment of the present invention, it was found that the Slit2 protein may induce reverse-aging or anti-aging of senescent cells, preferably mid-old cells.

In another aspect, the composition may be used for reverse-aging or anti-aging of senescent cells.

As used herein, the term “young cells” refers to cells that have not undergone aging and maintain proliferation ability of normal cells. As used herein, the term “young cells” may be used interchangeably with “normal cells” in terms of aging.

In the present invention, the young cells may have one or more of the following characteristics:
(1) increased expression of Slit2 compared to senescent cells; and
(2) decreased expression of serum amyloid A1 (SAA1) compared to senescent cells.

In addition, the young cells may have one or more of the following characteristics:
(1) negative for SA-β-Gal staining; and
(2) decreased expression of p16^{INK4A} compared to old cells.

In addition, the young cells may have one or more of the following characteristics:
(1) decreased expression of p53 and p21 (CDKN1A) compared to senescent cells;
(2) decreased cell size by 50% or less compared to senescent cells;
(3) increased cell growth and division rate by more than two times compared to senescent cells;
(4) decreased expression of inflammation-related genes IL1b, CCL2, CCL5, and IL6 compared to senescent cells;
(5) cell division cycle of 1 day or less.

As used herein, the term “senescent cells” refers to cells that have undergone aging or are undergoing aging. The senescent cells are in a state of proliferation inhibition or have a deteriorated cell proliferation ability, and the senescent cells may have the characteristic of secreting various inflammatory factors through changes in metabolic pathways. The inflammatory factors are known to promote aging of surrounding normal cells and tissues, ultimately causing diseases related to cellular aging and inducing individual aging.

In the present invention, the senescent cells may have one or more of the following characteristics.
(1) Decreased expression of Slit2 compared to young cells; and
(2) Increased expression of serum amyloid A1 (SAA1) compared to young cells.

In addition, the senescent cells may have one or more of the following characteristics:
(1) Increased expression of p53 and p21 (CDKN1A) compared to young cells;
(2) Increased cell size by more than two times compared to young cells;
(3) Decreased cell growth and division rate by less than 50% compared to young cells;
(4) Increased expression of inflammation-related genes IL1b, CCL2, CCL5, and IL6 compared to young cells; and
(5) Cell division cycle of 4 days or longer (cell division cycle of 16 days or longer in old cells).

In the present invention, the increase or decrease of the characteristics may be considered based on cells that maintain normal proliferative ability, i.e., young cells.

In the present invention, the senescent cells may be classified into “mid-old cells” and “old cells” depending on whether or not SA-β-Gal staining is performed or the expression level of p16 INK4A.

In the present invention, the senescent cells that have the characteristics of the senescent cells and are negative for SA-β-Gal staining, and/or have a similar expression level of p16^{INK4A} to that of young cells may be classified as mid-old cells.

In one embodiment of the present invention, it was found that Slit2 protein can induce reverse-aging or anti-aging of particularly mid-old cells.

Therefore, in the present invention, the senescent cells may be mid-old cells.

As used herein, the term “mid-old cells” refers to senescent cells that are negative for SA-β-Gal staining and/or have reduced p16^{INK4A} expression compared to old cells, among senescent cells, and are cells having reduced cell proliferation ability.

In the present invention, the mid-old cells may have any one or more of the following characteristics:
(1) negative for SA-β-Gal staining; and
(2) reduced p16^{INK4A} expression compared to old cells.

In addition, in the present invention, the mid-old cells may have any one or more of the following characteristics.
(1) Increased expression of p53 and p21 (CDKN1A) compared to young cells and decreased expression of p53 and p21 (CDKN1A) compared to old cells;
(2) Increased cell size by more than 2 times compared to young cells and decreased cell size by less than 50% compared to old cells;
(3) Decreased cell growth and division rate by less than 50% compared to young cells, and increased cell growth and division rate by more than 2 times compared to old cells;
(4) Increased expression of IL1b, CCL2, and CCL5 among inflammation-related genes compared to young cells and old cells;
(5) Increased expression of IL6 among inflammation-related genes compared to young cells;
(6) Decreased expression of IL6 among inflammation-related genes compared to old cells; and
(7) Cell division cycle of 4 days or more and 6 days or less.

As used herein, the term “old cells” refers to senescent cells that are positive for SA-β-Gal staining and/or have increased p16^{INK4A} expression compared to mid-old cells and are cells having inhibited cell proliferation ability.

In the present invention, the old cells may have any one or more of the following characteristics:
(1) positive for SA-β-Gal staining; and
(2) increased p16^{INK4A} expression compared to young cells and mid-old cells.

In addition, in the present invention, the old cells may have any one or more of the following characteristics.
(1) Increased expression of p53 and p21 (CDKN1A) compared to young cells and mid-old cells;
(2) Increased cell size by more than 2 times compared to young cells and mid-old cells;
(3) Decreased cell growth and division rate by less than 50% compared to young cells and mid-old cells;
(4) Increased expression of IL1b, CCL2, and CCL5 among inflammation-related genes compared to young cells;
(5) Decreased expression of IL6 IL1b, CCL2 and CCL5, among inflammation-related genes compared to mid-old cells;
(6) Increased expression of IL6 compared to old cells and mid-old cells; and
(7) Cell division cycle of 16 days or more.

In certain embodiments, the young cells may be anti-aged, the old cells may be reverse-aged, and the mid-old cells may be reverse-aged and/or anti-aged.

In the present invention, the inflammation-related gene may be, for example, selected from the group consisting of IL1b, CCL2, CCL5, SAA1, CXCL12, and IL6, but is not limited thereto.

As used herein, the term “p53” refers to an isoform of a protein encoded by a homologous gene in various organisms, such as TP53 (human) and Trp53 (mouse). This homolog (originally considered to be a single protein and often referred to as a “single protein”) is important in multicellular vertebrates in preventing cancer formation (Surget S, et al. (December 2013). OncoTargets and Therapy. 7: 57-68). As such, p53 has been described as “the guardian of the genome” because of the role thereof in preventing genomic mutations and preserving stability (Read AP, Strachan T (1999). Human molecular genetics 2. New York: Wiley. ISBN 978-0-471-33061-5). Therefore, TP53 is classified as a tumor suppressor gene.

As used herein, the term “p21” refers to a tumor suppressor protein encoded by the CDKN1A gene. p21 is important in preventing cancer as a tumor suppressor in the cell cycle of multicellular organisms. It is also known as cyclin-dependent kinase inhibitor 1 or CDK-interacting protein 1 (Cip 1). It inhibits cyclin-dependent kinase, thereby arresting the cell cycle.

As used herein, the term “p16^{INK4A}”, which is also known as p16, is a protein that delays cell division by slowing down cell cycle progression from the G1 phase to the S phase. Therefore, it acts as a tumor suppressor and is encoded by the CDKN2A gene.

As used herein, the term “senescence associated β-galactosidase (SA-β-Gal)” is a hydrolase that catalyzes the hydrolysis of β-galactosides into monosaccharides only in senescent cells. It is considered a biomarker of cellular aginge along with p16^{INK4A} (Hall B M, et al. (2016). Aging. 8 (7): 1294-1315). When the beta-galactosidase assay was performed at pH 6.0, it was observed that only old cells were stained.

As used herein, the term “IL8 [(Interleukin 8) or chemokine (C-X-C motif) ligand 8), CXCL8]” is a chemokine produced by macrophages and other cell types such as epithelial cells, airway smooth muscle cells, and endothelial cells. IL8 is a key mediator associated with inflammation that plays an important role in neutrophil recruitment and neutrophil degranulation. For example, IL8 has been cited as a proinflammatory mediator in gingivitis and psoriasis. IL8 secretion is increased by oxidant stress, which induces the recruitment of inflammatory cells and induces further increases in oxidant stress mediators, serving as a key parameter of localized inflammation.

As used herein, the term “interleukin 1b (IL1b)” is a key cytokine that regulates inflammation and immune responses to infection. Among the IL-1 family, IL-1b is known to be involved in chronic inflammatory diseases and autoimmune symptoms.

As used herein, the term “CCL2 (chemokine (C-C motif) ligand 2)” refers to a small cytokine belonging to the CC chemokine family. CCL2 recruits monocytes, memory T cells, and dendritic cells into sites of inflammation created by tissue damage or infection (Carr M W, et al. (April 1994). Proceedings of the National Academy of Sciences of the United States of America. 91 (9): 3652-6).

As used herein, the term “CCL5 (chemokine (C-C motif) ligand 5)” refers to an 8 kDa protein classified as a chemotactic cytokine or chemokine, and CCL5 is chemotactic for T cells, eosinophils, and basophils, and plays a role in recruiting leukocytes to the site of inflammation.

As used herein, the term “CXCL1 (chemokine (C-X-C motif) ligand 1)” refers to a small peptide belonging to the CXC chemokine family, which is expressed at a higher level during an inflammatory response and contributes to the inflammatory process (Silva R L, et al. (September 2017) Neurobiology of Disease. 105: 109-116).

As used herein, the term “CXCL2 (chemokine (C-X-C motif) ligand 2)” refers to a small cytokine belonging to the CXC chemokine family, and like related chemokines, CXCL2 is also a potent neutrophil chemoattractant and is involved in many immune responses, including wound healing, cancer metastasis, and angiogenesis (Al-Alwan L A, et al. (September 2013). Journal of Immunology. 191 (5): 2731-41).

As used herein, the term “IL6 (interleukin 6)” refers to an interleukin that acts as a proinflammatory cytokine and an anti-inflammatory myokine. IL6 is present on the cell surface and in intracellular compartments and induces intracellular signaling cascades that cause the production of inflammatory cytokines.

As used herein, the term “CXCL12 (C-X-C motif chemokine 12)”, which is also called “stromal cell-derived factor 1 (SDF1)”, is a chemokine protein encoded by the CXCL12 gene on chromosome 10 in humans. CXCL12 is expressed ubiquitously in many types of tissues and cells. CXCL12 is a potent chemotactic receptor for lymphocytes. CXCR4, which was previously called “LESTR” or “fusin”, is the receptor for CXCL12 (Bleul C C, et al. (September 1996). The Journal of Experimental Medicine. 184 (3): 1101-9). Actions based on CXCR4 and CXCL12 have inhibitory effects on neuroinflammation in mice, rats, and humans.

As used herein, the term “serum amyloid A1 (SAA1)” is a protein encoded by the SAA1 gene in humans, which is a major acute-phase protein produced primarily in hepatocytes in response to infection, tissue damage, and malignancy (Gabay C, Kushner I (February 1999). The New England Journal of Medicine. 340 (6): 448-54). It is well known that elevated plasma concentrations of SAA1 are associated with a number of inflammatory conditions and SAA1 is a clinical marker and reliable biomarker for inflammatory diseases, chronic metabolic disorders and advanced malignancies (Malle E, et al. (January 2009). Cellular and Molecular Life Sciences. 66 (1): 9-26).

In one embodiment of the present invention, the expression patterns of SAA1, Slit2, and p16^{INK4A} were found in in vivo aged tissue (75 years or older). The expression of SAA1 was increased and the expression of Slit2 was decreased in aged tissue, which indicates that senescent cells were mainly present therein. In addition, there were almost no cells expressing p16^{INK4A}, which indicates that mid-old cells were more predominantly present than old cells.

In another embodiment of the present invention, it was found that when mid-old cells were treated with Slit2 protein, they were reverse-aged or anti-aged. It will be obvious to those skilled in the art that diseases related to cellular aging could be prevented or treated by reverse-aging or anti-aging of such cells.

In another aspect, the present invention provides a pharmaceutical composition for preventing or treating a cellular aging-related disease containing Slit2 protein.

In another aspect, the present invention provides the use of Slit2 protein for preventing or treating a cellular aging-related disease.

In another aspect, the present invention provides the use of Slit2 protein for the preparation of a pharmaceutical composition for preventing or treating a cellular aging-related disease.

In another aspect, the present invention provides a method for preventing or treating a cellular aging-related disease, including administering Slit2 protein to a subject.

As used herein, the term “cellular aging-related disease” refers to a disease or disorder caused by cellular aging, which is a disease or disorder that occurs due to a decrease in cell function or a change in cell morphology caused by cellular aging.

Such a disease or disorder related to cellular aging may vary depending on the aged cells, but examples thereof include: neurodegenerative diseases or disorders; cardiovascular diseases or disorders; metabolic diseases or disorders; pulmonary diseases or disorders; inflammatory or autoimmune diseases or disorders; transplant-related diseases or disorders; eye diseases or disorders; proliferative diseases or disorders; side effects of chemotherapy and radiotherapy; geriatric diseases or disorders; fibrotic diseases or disorders; skin diseases or disorders; and diseases or disorders caused by stem cellular aging.

In a specific example, when the cellular aging is fibroblast aging, it may cause chronic skin damage (such as psoriasis, eczema, wrinkles, moles, scars, or keloids), skin atrophy, obesity, diabetes, degenerative neurological diseases, osteoarthritis, osteoporosis, atherosclerosis, age-related eye diseases (such as cataracts, glaucoma, presbyopia, or macular degeneration), or fibrosis (such as pulmonary fibrosis, cystic fibrosis, renal fibrosis, liver fibrosis, oral submucosal fibrosis, cardiac fibrosis, or pancreatic fibrosis).

Examples of the neurodegenerative diseases or disorders include cognitive impairment, Alzheimer's disease/dementia, Parkinson's disease, motor neuron dysfunction, Huntington's disease, and the like.

Examples of the cardiovascular diseases or disorders include atherosclerosis, cardiac diastolic dysfunction, aortic aneurysm, cerebral aneurysm, angina pectoris, arrhythmia, cardiomyopathy, congestive heart failure, coronary artery disease, myocardial infarction, endocarditis, hypertension, carotid artery disease, peripheral artery disease, cardiac load resistance, cardiac fibrosis, heart attack (coronary artery thrombosis), dyslipidemia (hypertriglyceridemia/hyperlipidemia, hypercholesterolemia), mitral valve prolapse, stroke, cerebrovascular disease, and the like.

Examples of the metabolic diseases or disorders include diabetes, dyslipidemia, obesity, metabolic syndrome, diabetic ulcer, insulin resistance, hepatic steatosis (steatohepatitis), and the like.

Examples of the lung diseases or disorders include chronic obstructive pulmonary disease, pulmonary fibrosis, idiopathic pulmonary fibrosis, emphysema, obliterative bronchiolitis, asthma, cystic fibrosis, bronchiectasis, and the like.

Examples of the inflammatory or autoimmune diseases or disorders include osteoarthritis and degenerative joint diseases, rheumatoid arthritis, inflammatory bowel disease, mucositis (oral mucositis), and the like.

Examples of the eye diseases or disorders include macular degeneration, cataracts, glaucoma, visual impairment, presbyopia, and the like.

Examples of the proliferative diseases or disorders include various cancers and cancer metastases, benign prostatic hyperplasia, and the like.

Examples of the chemotherapy and radiotherapy side effects include fatigue/malaise/low physical activity, gastrointestinal toxicity, peripheral neuropathy, hematologic toxicity, hepatotoxicity, cardiac toxicity, alopecia, pain, mucositis, fluid retention, dermatological toxicity, and the like.

Examples of the geriatric diseases or disorders include osteoporosis, kyphosis, hydrocele, herniated disc, hepatic disorders, renal dysfunction (renal failure, glomerulosclerosis, glomerulonephritis), weakness, urinary incontinence, gait disturbance, alopecia, hearing loss, muscle fatigue, skin conditions, skin nevi, sarcopenia, skin wound healing, and other geriatric diseases induced by aging (e.g., diseases/disorders resulting from smoking, high-fat/high-sugar diets, and environmental factors).

Examples of the fibrotic diseases or disorders include pulmonary fibrosis, cystic fibrosis, renal fibrosis, liver fibrosis, oral submucosal fibrosis, cardiac fibrosis, and pancreatic fibrosis.

Examples of the skin diseases or disorders include psoriasis, eczema, wrinkles, loss of elasticity, pruritus, paresthesia, dystonia, erythroderma, lichen planus, lichenoid dermatosis, nevi, rash, urticaria, atopic dermatitis, eosinophilic dermatosis, reactive neutrophilic dermatosis, pemphigoid, pemphigoid, immune bullous dermatosis, fibrous histiocytoma, cutaneous lymphoma, cutaneous lupus, hyperpigmentation, scars, keloids, strawberry nose, vitiligo, ichthyosis, dermatomyositis, lentigines, blemishes, freckles, liver spots, actinic keratosis, and the like. In the present invention, the cellular aging-related disease preferably includes at least one disease selected from the group consisting of cancer, fibrosis, Alzheimer's diseases, arthritis, atherosclerosis, and retinal degeneration, but is not limited thereto.

As used herein, the terms “prevention” and “treatment” should be interpreted in the broadest sense, and the term “prevention” means preventing one or more of the clinical symptoms of a disease from progressing in a patient who may be exposed to or susceptible to a disease but has not yet experienced or shown symptoms of the disease, and the term “treatment” means any act of preventing or reducing the development of a disease or one or more of clinical symptoms thereof, but is not limited thereto.

The pharmaceutical composition for preventing or treating a cellular aging-related disease according to the present invention may further contain a pharmaceutically acceptable carrier, and may be formulated along with the carrier.

As used herein, the term “pharmaceutically acceptable carrier” refers to a carrier or diluent that does not impair the biological activities or properties of the administered compound and does not irritate an organism. Pharmaceutically acceptable carriers for compositions formulated into liquid solutions are sterilized and biocompatible, and examples thereof include saline, sterile water, Ringer's solution, buffered saline, albumin injection solutions, dextrose solutions, maltodextrin solutions, glycerol, and mixtures of one or more thereof. If necessary, other conventional additives such as antioxidants, buffers and bacteriostatic agents may be added. In addition, injectable solutions such as aqueous solutions, suspensions and emulsions, pills, capsules, granules, or tablets may be formulated by further adding diluents, dispersants, surfactants, binders and lubricants, but is not limited thereto.

The pharmaceutical composition for preventing or treating a cellular aging-related disease containing the compound and a pharmaceutically acceptable carrier according to the present invention may be applied to any formulation containing the compound as an active ingredient and may be prepared as an oral or parenteral formulation. The pharmaceutical formulation of the present invention includes, but is not limited to, a form suitable for inhalation, oral, rectal, nasal, topical (including buccal and sublingual), subcutaneous, vaginal or parenteral (including intramuscular, subcutaneous and intravenous) administration, or a form suitable for administration by injection.

The formulation for oral administration containing the composition of the present invention as an active ingredient may be formulated as, for example, a tablet, troches, lozenge, aqueous or oily suspension, prepared powder or granule, emulsion, hard or soft capsule, syrup or elixir. In order to prepare the composition into a formulation such as tablet and capsule, the formulation may contain a binder such as lactose, saccharose, sorbitol, mannitol, starch, amylopectin, cellulose or gelatin, an excipient such as dicalcium phosphate, a disintegrant such as corn starch or sweet potato starch, a lubricant such as magnesium stearate, calcium stearate, sodium stearyl fumarate or polyethylene glycol wax, and the capsule, in addition to the above-mentioned substances, may further contain a liquid carrier such as a fatty oil. The formulation for parenteral administration containing the composition of the present invention as an active ingredient may be formulated as a form for injection such as subcutaneous injection, intravenous injection or intramuscular injection, as a suppository, or a spray such as an aerosol that can be inhaled through the respiratory tract. The formulation for injection may be prepared by mixing water with a stabilizer and a buffer to obtain a solution or suspension and injecting the solution or suspension into an ampoule or vial for single (unit) administration. The formulation for administration as a suppository may be prepared as a composition for rectal administration such as a suppository or enema containing a conventional suppository base such as cocoa butter or another glyceride. When formulated as a spray, such as an aerosol, a propellant or the like may be blended with an additive so that the water-dispersed concentrate or wet powder is dispersed, but is not limited thereto.

In another aspect, the present invention is directed to an anti-aging cosmetic composition containing Slit2 protein.

In another aspect, the present invention is directed to the use of an anti-aging cosmetic composition.

In another aspect, the present invention is directed to the use of Slit2 protein for the preparation of an anti-aging cosmetic composition.

In another aspect, the present invention is directed to the use of Slit2 protein for the preparation of an anti-aging cosmetic composition and/or a reverse-aging cosmetic composition containing Slit2 protein.

The anti-aging cosmetic composition may be used for various functions and purposes based on the reverse-aging effect of the Slit2 protein, for example, for moisturizing the skin, reinforcing the skin barrier, soothing the skin, preventing or ameliorating skin damage, or preventing or reversing aging, or improving skin condition, but is not limited thereto. “Skin condition improvement” is used in a comprehensive sense including not only the improvement of the observed skin condition, but also factors that directly and indirectly affect skin health.

The anti-aging cosmetic composition may further contain a cosmetically acceptable excipient or carrier, and may further contain any of various known additives depending on the type of the formulation, for example, an additive selected from the group consisting of a carrier, an emulsifier, a moisturizer, a surfactant, a chelating agent, an antioxidant, a sterilizer, a stabilizer, and any combination thereof.

The carrier may be, for example, an animal oil, vegetable oil, wax, paraffin, starch, tragacanth, a cellulose derivative, polyethylene glycol, silicone, bentonite, silica, talc, zinc oxide, lactose, aluminum hydroxide, calcium silicate, a polyamide powder, water, ethanol, isopropanol, ethyl carbonate, ethyl acetate, benzyl alcohol, benzyl benzoate, propylene glycol, 1,3-butyl glycol oil, glycerol fatty esters, liquid diluting agents, suspensions such as ethoxylated isostearyl alcohol, polyoxyethylene sorbitol ester and polyoxyethylene sorbitan ester, microcrystalline cellulose, aluminum metahydroxide, agar, fatty alcohol sulfate, fatty alcohol ether sulfate, sulfosuccinic acid monoester, isethionate, imidazolinium derivatives, methyltaurate, sarcosinate, fatty acid amide ether sulfate, alkylamidobetaine, fatty alcohol, fatty acid glycerides, fatty acid diethanolamide, linoline derivatives, or ethoxylated glycerol fatty acid esters, or combinations thereof, but are not limited thereto.

The emulsifier may include, but is not limited to, liquid paraffin, cetyl octanoate, stearic acid, and the like.

The moisturizing agent may include, but is not limited to, glycerin, butylene glycol, propylene glycol, dipropylene glycol, pentylene glycol, hexylene glycol, polyethylene glycol, sorbitol, and the like.

The chelating agent may include, but is not limited to, ethylene-diamine-tetraacetic acid (EDTA), α-hydroxy fatty acids, lactoferrin, α-hydroxy acid, citric acid, lactic acid, malic acid, bilirubin, biliverdin, and the like.

The antioxidant may include, but is not limited to, butylated hydroxyanisole, butylated hydroxytoluene, or propyl gallate.

In addition, in the present invention, the anti-aging cosmetic composition may further contain one or more active ingredients, in addition to the Slit2 protein of the present invention.

In the present invention, the anti-aging cosmetic composition may be prepared and used in various known formulations depending on the intended use and method of use. For example, the anti-aging cosmetic composition according to the present invention may be in the form of, but is not limited to, a toner, a facial lotion, a body lotion, a nourishing cream, a moisturizing cream, an eye cream, an essence, a cosmetic ointment, a spray, a gel, a pack, a sunscreen, a makeup base, a foundation, a powder, a cleansing cream, a cleansing lotion, a cleansing oil, a cleansing foam, a soap, or a body wash.

In the anti-aging cosmetic composition according to the present invention, when the skin is the scalp or hair, the formulation may be a hair tonic, a hair conditioner, a hair essence, a hair lotion, a hair nutritional lotion, a hair shampoo, a hair rinse, a hair treatment, a hair cream, a hair nutritional cream, a hair moisturizing cream, a hair massage cream, a hair wax, a hair aerosol, a hair pack, a hair nutritional pack, a hair soap, a hair cleansing foam, a hair oil, a hair drying agent, a hair preservation treatment agent, a hair dye, a hair waving agent, a hair bleaching agent, a hair gel, a hair glaze, a hair dressing agent, a hair lacquer, a hair moisturizer, a hair mousse or a hair spray, but is not limited thereto.

The Slit2 protein, which is an active ingredient of the anti-aging cosmetic composition, and the additive may be contained in a cosmetically acceptable amount, and may preferably be contained within the scope of regulations established by each country.

In the present invention, the anti-aging cosmetic composition may be contained in functional cosmetics. The standard for the functional cosmetics may follow the Korean “Functional Cosmetic Standards and Test Methods Notice of the Ministry of Food and Drug Safety,” and may have functions such as, for example, skin whitening, wrinkle improvement, hair color change, hair loss relief or prevention, acne relief, and ultraviolet protection, but is not limited thereto.

Previously reported anti-aging methods have side effects that may cause tumor formation and cancer progression. In an attempt to minimize the side effects, according to the present invention, it was found that, when young cells and senescent cells are co-cultured, the senescent cells are reverse-aged and the young cells highly express Slit2 genes, and it was found that, when senescent cells are treated with the Slit2 protein, the senescent cells are reverse-aged.

Therefore, in another aspect, the present invention is directed to a method for reverse-aging or anti-aging of senescent cells including treating the senescent cells with the Slit2 protein.

Therefore, in the present invention, the senescent cells may be mid-old cells.

In addition, in the present invention, when the senescent cells are treated with Slit2 protein, they may have one or more of the following characteristics.
(1) Increased expression of Slit2;
(2) Decreased expression of serum amyloid A1 (SAA1);
(3) Decreased expression of p53 and p21;
(4) Decreased cell size;
(5) Increased cell growth; and
(6) Decreased expression of inflammatory genes.

In addition, in the present invention, when the senescent cells are treated with Slit2 protein, they may have all of the following characteristics.

In the present invention, the inflammation-related gene may be, for example, selected from the group consisting of IL1b, CCL2, CCL5, SAA1, CXCL12, and IL6, but is not limited thereto.

In the present invention, the Slit2 protein may be a Slit2-N protein or a Slit2-C protein, and preferably, the Slit2 protein may be a Slit2-N protein.

In the present invention, the Slit2 protein may include a sequence selected from the group consisting of NCBI reference sequences NP_004778.1, NP_001276064.1, NP_001276065.1, XP_005248268.1, XP_006714049.1, XP_011512211.1, XP_016864334.1, and XP_011512212.2, and preferably, NP_004778.1.

In the present invention, the Slit2-N protein may be a recombinant human Slit2-N protein including a sequence represented by SEQ ID NO. 1.

In the present invention, the Slit2 protein is used to encompass not only natural proteins but also recombinant proteins and fragments thereof, as long as the activity thereof is maintained.

In an embodiment of the present invention, a recombinant human Slit2-N protein including a sequence represented by SEQ ID NO. 1 was used (purchased from PEPROTECH, catalog number 150-11, Source: HEK293 cells), but is not limited thereto.

In the present invention, mid-old cells may be treated with the recombinant human Slit2-N protein at 10 ng/mL to 1,000 ng/mL, preferably 50 ng/mL to 500 ng/mL, and more preferably 100 ng/mL to 200 ng/mL, and in an embodiment of the present invention, mid-old cells were treated with 100 ng/mL of the recombinant human Slit2-N protein, but is not limited thereto.

In the present invention, the mid-old cells may be treated with the recombinant human Slit2-N protein once for 1 to 8 days, preferably once for 2 to 6 days, and more preferably every two days, and in an embodiment of the present invention, the mid-old cells were treated with the recombinant human Slit2-N protein every two days, but the present invention is not limited thereto.

In the present invention, the treatment period of the recombinant human Slit2-N protein may be determined at 4, 8, 12, or 16 days, preferably 8 days, after the mid-old cells are treated with the recombinant human Slit2-N protein. In an embodiment of the present invention, the treatment period of the recombinant human Slit2-N protein may be determined at 8 days after treating the recombinant human Slit2-N protein, but it is not limited thereto.

In another embodiment of the present invention, mid-old cells are treated with recombinant human Slit2-N protein and then the expression patterns of p53 and p21 are determined. The mid-old cells may be treated with the recombinant human Slit2-N protein at 5 ng/mL to 100 ng/mL, preferably 10 ng/mL to 40 ng/mL, and in the embodiment of the present invention, the mid-old cells may be treated with 10 ng/mL to 40 ng/mL of recombinant human Slit2-N protein, but is not limited thereto.

In the present invention, the mid-old cells may be treated with recombinant human Slit2-N protein for 1 day, 2 days, 3 days, 4 days, 5 days, or 6 days, preferably 2 days, and in the embodiment of the present invention, the mid-old cells may be treated with the recombinant human Slit2-N protein for 2 days, but is not limited thereto.

In another embodiment of the present invention, the mid-old cells were treated with recombinant human Slit2-N protein, and cell proliferation was compared between the mid-old cells and the control group. The mid-old cells are treated with recombinant human Slit2-N protein at 10 ng/mL to 1,000 ng/mL, preferably 50 ng/mL to 500 ng/mL, and in the embodiment of the present invention, the mid-old cells are treated with 100 ng/mL of the recombinant human Slit2-N protein, but the present invention is not limited thereto.

In the present invention, the mid-old cells may be treated with recombinant human Slit2-N protein for 1 day, 5 days, 10 days, 15 days, 20 days, 25 days, 30 days, 35 days, 40 days, 45 days, or 50 days, preferably 20 days, and in the embodiment of the present invention, the mid-old cells may be treated with the recombinant human Slit2-N protein for 20 days, but the present invention is not limited thereto.

In the present invention, the mid-old cells are treated with the recombinant human Slit2-N protein and the cell count is measured every 1 day, every 2 days, every 3 days, every 4 days, every 5 days, every 6 days, or every 7 days, preferably every 4 days. In an embodiment of the present invention, the mid-old cells are treated with the recombinant human Slit2-N protein and the cell count is measured every 4 days, but the present invention is not limited thereto.

In another embodiment of the present invention, aged mice were treated with the recombinant mouse Slit2-N protein and the activity distance was compared between the mice and the control group (SEQ ID NO: 27). The recombinant mouse Slit2-N protein may be administered intraperitoneally at 0.5 μg/mouse, 1 μg/mouse, 1.5 μg/mouse, 2 μg/mouse, 2.5 μg/mouse, or 3 μg/mouse, preferably 2 μg/mouse, and in an embodiment of the present invention, the recombinant mouse Slit2-N protein was administered intraperitoneally at 2 μg/mouse, but the present invention is not limited thereto.

In the present invention, the mice may be treated with recombinant mouse Slit2-N protein for 1 week, 2 weeks, 3 weeks, 4 weeks, or 5 weeks, preferably for 3 weeks, and in an embodiment of the present invention, the mice were treated with recombinant mouse Slit2-N protein for 3 weeks, but the present invention is not limited thereto.

In the present invention, the mice may be treated with recombinant mouse Slit2-N protein once a week, twice a week, three times a week, four times a week, five times a week, six times a week, or seven times a week, preferably twice a week, and in an embodiment of the present invention, the mice may be treated with the recombinant mouse Slit2-N protein twice a week, but the present invention is not limited thereto.

In one embodiment of the present invention, it was found that, when young cells secrete Slit2 protein and young cells and mid-old cells are co-cultured, the Slit2 protein is secreted from the young cells and the mid-old cells are reverse-aged or anti-aged.

In the present invention, the step of treating the senescent cells with the Slit2 protein may be performed by co-culturing the senescent cells with young cells that express and/or secrete the Slit2 protein.

As used herein, the term “co-culture” has long been used to study interactions between cell populations, and is the basis of all types of cell-cell interaction studies. At a basic level, co-culture is a cell culture set in which two or more different cell populations grow in some degree of contact with each other. The co-culture is used to study natural interactions between populations, improve the success of cultures for specific populations, or establish synthetic interactions between populations. Examples of study on natural interactions between populations include, but are not limited to, infection studies, other natural interactions, experimental models of natural systems such as artificial tissues, and creating biomimetic environments.

The co-culture of the present invention may be performed by co-culturing mid-old cells and young cells at a ratio of 2:1 or 1:2, preferably 1:1, and in the embodiment of the present invention, co-culturing was performed by co-culturing mid-old cells and young cells at a ratio of 1:1, but the present invention is not limited thereto.

In the step of obtaining the reverse-aged cells of the present invention, the reverse-aged cells may be obtained 20 days after, 30 days after, 40 days after, or 50 days after co-culturing, preferably 30 days after co-culturing, and in the embodiment of the present invention, the reverse-aged cells may be obtained 30 days after co-culturing, but the present invention is not limited thereto.

Hereinafter, the present invention will be described in more detail with reference to examples. However, it will be obvious to those skilled in the art that these examples are provided only for illustration of the present invention, and should not be construed as limiting the scope of the present invention.

### Example 1: Characterization of Young Cells, Mid-Old Cells, and Old Cells

### 1.1 Confirmation of Morphology

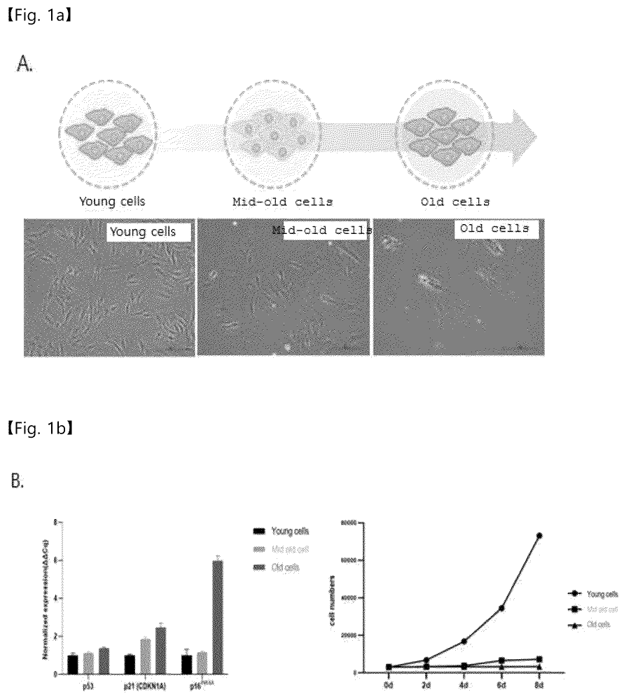

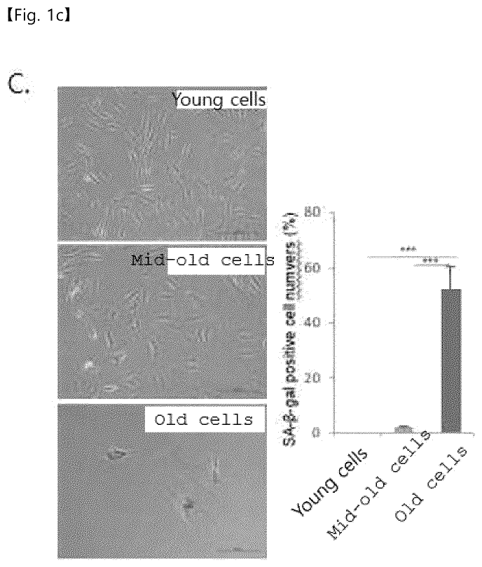

Normal fibroblasts were sub-cultured in a culture dish at a ratio of 70% and whether or not cell division occurred was determined. Cells with a cell division cycle of 1 day or less were classified as young cells, cells with a cell division cycle of 4 to 6 days or less were classified as mid-old cells, and cells with a cell division cycle of 16 days or more were classified as old cells. The morphologies of young cells, mid-old cells, and old cells were observed. It was found that cells present in the human body enter the senescence as they undergo aging, that is, repeatedly divide, and the size of the cytoplasm and nucleus gradually increases (FIG. 1A). It was found that mid-old cells have a cell size more than twice that of young cells, and old cells have a cell size more than four times that of young cells.

1.2 Confirmation of Expression Patterns and Cell Growth of p53, p21 (CDKN1A), and p16^{INK4A}

It was confirmed that the expression of p53, p21 (CDKN1A), and p16^{INK4A} increased and the number of cells decreased as cells aged (FIG. 1B).

Similar to Example 1.1 above, normal fibroblasts were sub-cultured and the division rate was observed, and then young cells (cell division cycle of 1 day or less), mid-old cells (cell division cycle of 4 to 6 days) and old cells (cell division cycle of 16 days or longer) were classified based on the division cycle. RNA was extracted from the cells in the culture dish using an RNA extraction kit, cDNA was synthesized, and the expression of senescence marker genes (p53, p21 (CDKN1A), and p16^{INK4A}) was measured using real-time PCR using primers.

The expression of p53, p21 (CDKN1A), and p16^{INK4A}, which are marker genes for aging, was analyzed using real-time PCR to measure the level of expression depending on the type of cells, the level of expression was calculated by fold induction and the resulting expression levels in young cells, mid-old cells, and old cells were compared based on the expression level (the value of 1) in young cells. The result showed that p53 and p21 (CDKN1A) were found to be representative aging cell markers which increased by more than 2-fold in mid-old cells and old cells, while p16^{INK4A} was found to be a gene that was overexpressed only in old cells and was increased by more than 2-fold only in old cells. This suggests that p53 and p21 (CDKN1A) may be used as markers to distinguish between young cells and senescent cells (mid-old cells and old cells), and p16^{INK4A} may be used as a marker to distinguish between mid-old cells and old cells.

### 1.3 Confirmation of the Number of Cells Positive for SA-β-Gal

The numbers of young cells, mid-old cells, and old cells positive for SA-β-Gal, a marker of aging, were determined.

The cells were treated with a SA-β-Gal solution consisting of 1 mg mL⁻¹ of X-gal, 40×10⁻³ M citric acid/sodium phosphate (pH 5.8), 5×10⁻³ M potassium ferrocyanide, 5×10⁻³ M potassium ferricyanide, 150×10⁻³ M sodium chloride (NaCl), and 2×10⁻³ M magnesium chloride (MgCl₂) at 37° C. for 12 hours.

The number of old cells positive for SA-β-Gal was found to be increased compared to young cells and mid-old cells (FIG. 1C). This suggests that SA-β-Gal may be used as a marker to distinguish old cells from mid-old cells.

### 1.4 Confirmation of Expression Pattern of Inflammation-Related Genes

The expression patterns of inflammation-related genes (IL8, IL1b, CCL2, CCL5, CXCL1, CXCL2, SAA1, CXCL12, IL6) were determined in young cells, mid-old cells, and old cells.

RNA is extracted from each cell, cDNA is synthesized and real-time PCR is performed using IL8, IL1b, CCL2, CCL5, CXCL1, CXCL2, SAA1, CXCL12, and IL6 primers.

The primer sequences used in the real-time PCR are shown in Table 1 below.

TABLE 1
Primer sequences

| | | SEQ ID |
|---|---|---|
| Primer | Sequence | NO: |
| IL8 | F - CTG GCC GTG GCT CTC TTG | 2 |
| (CXCL8) | R - CCT TGG CAA AAC TGC ACC TT | 3 |
| IL1b | F - CTG TCC TGC GTG TTG AAA GA | 4 |
| | R - TTG GGT AAT TTT TGG GAT CTA CA | 5 |
| CCL2 | F - CTC TGC CGC CCT TCT GTG | 6 |
| | R - TGC ATC TGG CTG AGC GAG | 7 |
| CCL5 | F - ATG AAG GTC TCC GCG GCA GC | 8 |
| | R - CTA GCT CAT CTC CAA AGA GT | 9 |
| CXCL1 | F - CTT GCC TCA ATC CTG CAT C | 10 |
| | R - CCT TCT GGT CAG TTG GAT TTG | 11 |
| CXCL2 | F - TGC AGG GAA TTC ACC TCA AG | 12 |
| | R - TGA GAC AAG CTT TCT GCC CA | 13 |
| SAA1 | F - TTT CTG CTC CTT GGT CCT GG | 14 |
| | R - CTC TGG CAT CGG TGA TCA CT | 15 |
| CXCL12 | F - TGC CAG AGC CAA CGT CAA G | 16 |
| | R - CAG CCG GGC TAC AAT CTG AA | 17 |
| IL6 | F - AAT TCG GTA CAT CCT CGA CGG | 18 |
| | R - GGT TGT TTT CTG CCA GTG CC | 19 |

It was found that the expression of inflammation-related genes, except for IL6, increased in mid-old cells, compared to young cells (FIG. 1D and FIG. 1E). This suggests that reverse-aging and anti-aging may have an anti-inflammatory effect.

### Example 2: Confirmation of Characteristics of Mid-Old Cells In Vitro

### 2.1 Characteristics of Mid-Old Cells

Similar to Example 1.1 above, normal fibroblasts were subcultured and the division rate was observed, and then young cells (cell division cycle of 1 day or less), mid-old cells (cell division cycle of 4 to 6 days), and old cells (cell division cycle of 16 days or more) were classified based on the division cycle. RNA was extracted from the cells in the culture dish using an RNA extraction kit, cDNA was synthesized, and real-time PCR was performed using primers to measure the expression of Slit2 and SAA1 genes.

The primer sequences used in the real-time PCR are as shown in Table 2 below.

TABLE 2
Primer sequences

| | | SEQ ID |
|---|---|---|
| Primer | Sequence | NO: |
| Slit2 | F- GTC AAT GAC CTA CTG GCC TCA TTC | 20 |
| | R- TGA TTG CTA CAC ACC ATT TCG TTT C | 21 |

The expression of Slit2 and SAA1 was analyzed using real-time PCR to measure the level of expression depending on the type of cells, the level of expression was calculated by fold induction and the resulting expression levels in young cells, mid-old cells, and old cells were compared based on the expression level (the value of 1) in young cells. The result showed that mid-old cells exhibit an at least 8-fold increase in the SAA1 expression level compared to young cells, and old cells exhibit an at least 6-fold increase in the SAA1 expression level compared to in young cells. Slit2 exhibits an at least 4-fold decrease in the expression level in mid-old cells and old cells compared to in young cells. This suggests that, as aging progresses, the expression level of SAA1 increases and the expression level of Slit2 decreases, and that the expression levels of SAA1 and Slit2 may be used as markers of young and old cells.

### 2.2 Comparison in Gene Expression Patterns Between Young, Mid-Old, and Old Cells

The result of data analysis showed that p16^{INK4A} and SAA1 gene expression was decreased and Slit2 gene expression was increased in young cells;

p16^{INK4A} and Slit2 gene expression was decreased and SAA1 gene expression was increased in mid-old cells; and

Slit2 gene expression was decreased, p16 INK4A gene expression was increased, and SAA1 gene expression was decreased in old cells, compared to in mid-old cells (FIG. 3).

### Example 3: Confirmation of Presence of Mid-Old Cells in Aged Tissues In Vivo

Paraffin blocks were produced from normal parts (colon, lung) of young tissues (under 40 years old) and aged tissues (over 75 years old), and the expression of SAA1 and Slit2 was identified through immunohistochemical analysis. The paraffin blocks were treated with primary antibodies and were bound to the target substances, SAA1 and Slit2, and then treated with secondary antigens, which are chromogenic substances, and whether or not color was developed was determined.

The antibody sequences used are shown in Table 3 below.

TABLE 3
Antibody sequences

| | | SEQ ID |
|---|---|---|
| Antibody | Sequence | NO. |
| SAA1 | MKLLTGLVFCSLVLGVSSRSFFSELGEAFDGARDMWRA | 22 |
| | YSDMREANYIGSDKYFHARGNYDAAKRGPGGVWAAEAI | |
| | SDARENIQRFFGHGAEDSLADQAANEWGRSGKDPNHER | |
| | PAGLPEKY | |
| Slit2 | LYINSELQDFQKVPMQTGILPGCEPCHKKVCAHGTCQP | 23 |
| | SSQAGFTCECQEGWMGPLCDQRTNDPCLGNKCVHGTCL | |
| | PINAFSYSCKCLE | |

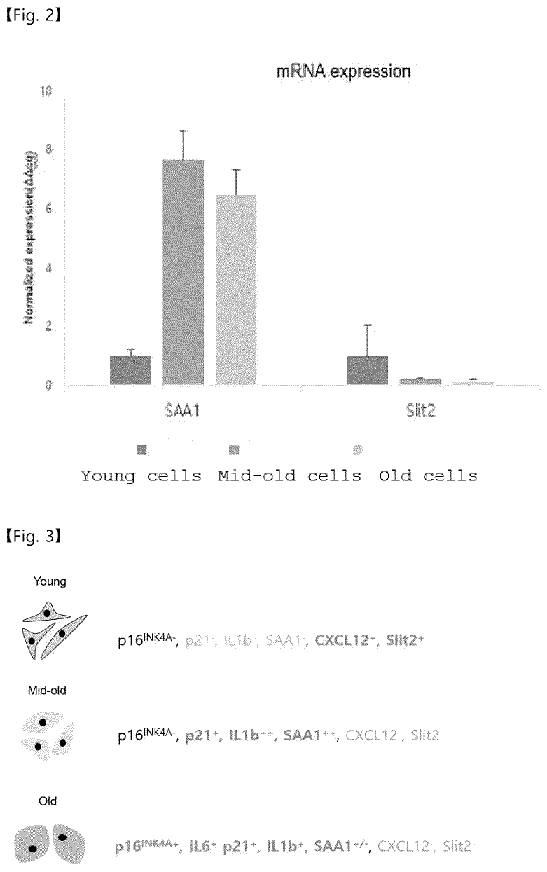

As a result, when p16^{INK4A} was analyzed as a target, almost no colored cells were observed in aged tissues (FIG. 4A). When Slit2 was analyzed as a target protein, almost no colored cells were observed in aged tissues, and when SAA1 was analyzed as a target protein, many colored cells were observed in aged tissues (FIG. 4B). This suggests that, in aged tissues, there were almost no old cells expressing p16^{INK4A}, a senescence marker, and mid-old cells expressing a high level of SAA1, but expressing a low level of Slit2 were dominantly present.

### Example 4: Co-Culture of Young Cells and Mid-Old Cells

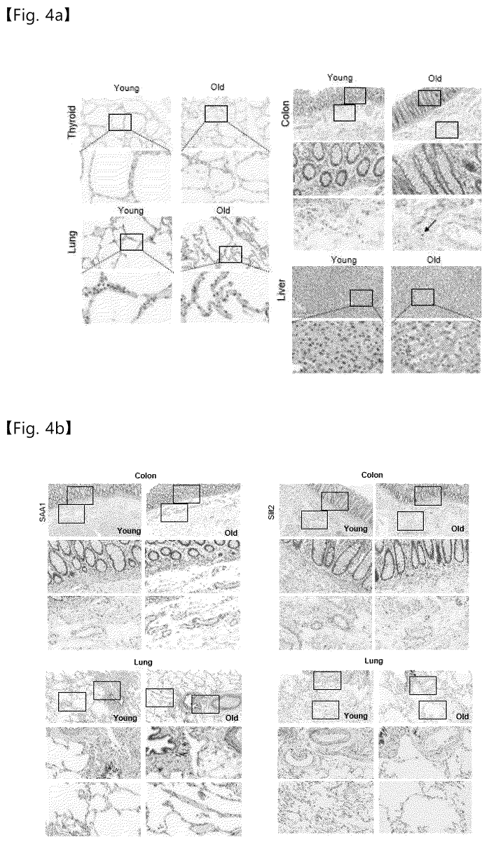

To determine whether or not the substance secreted by young cells induces the reverse aging of mid-old cells, mid-old cells and mid-old cells, and young cells and mid-old cells were co-cultured. Specifically, 1×10⁴ mid-old cells were seeded in the low chamber using Transwell, and 1 day later, 1×10⁴ mid-old cells or young cells were seeded in the upper chamber and co-cultured for 30 days, and the upper chamber was replaced once every 7 days (FIG. 5A). After co-culture for 30 days, the cell count in the low chamber was measured and the result showed that the cell growth was faster and the cell count was higher in the co-culture of young cells and mid-old cells than in the co-culture of mid-old cells and mid-old cells (FIG. 5B).

This suggests that the substance secreted from young cells improves the cell growth of mid-old cells and promotes cell division. The increase in cell growth and promotion of cell division are characteristics of young cells, which means that aging of the mid-old cells had been reversed.

### Example 5: Confirmation of Presence of Slit2 in Substances Secreted from Young Cells

In the same manner as Example 2.1, the expression of Slit2 was analyzed through real-time PCR in young cells, mid-old cells, and old cells.

In addition, the expression of Slit2 in young cells, mid-old cells, and old cells was detected through ELISA analysis.

The antibody sequences used are shown in Table 4 below.

TABLE 4
Antibody sequence

| | | SEQ ID |
|---|---|---|
| Antibody | Sequence | NO: |
| Slit2 | SPPMVLPRTSPCDNEDCQNGAQCIVRINEPICQCLPGYQGEK | 24 |
| | CEKLVSVNFINKESYLQIPSAKVRPQTNITLQIATDEDSGIL | |
| | LYKGDKDHIAVELYRGRVRASYDTGSHPASAIYSVETINDGN | |
| | FHIVELLALDQSLSLSVDGGNPKIITNLSKQSTLNEDSPLYV | |
| | GGMPGKSNVASLRQAPGQNGTSFHGCIRNLYINSELQDFQKV | |
| | PMQTGILPGCEPCHKKVCAHGTCQPSSQAGFTCECQEGWMGP | |
| | LCDQRTNDPCLGNKCVHGTCLPINAFSYSCKCLEGHGGVLCD | |
| | EEEDLENPCQAIKCKHGKCRLSGLGQPYCECSSGYTGDSDRE | |
| | ISCRGERIRDYYQKQQGYAACQTTKKVSRLECRGGCAGGQCC | |
| | GPLRSKRRKYSFECTDGSSFVDEVEKVVKCGCTRCV | |

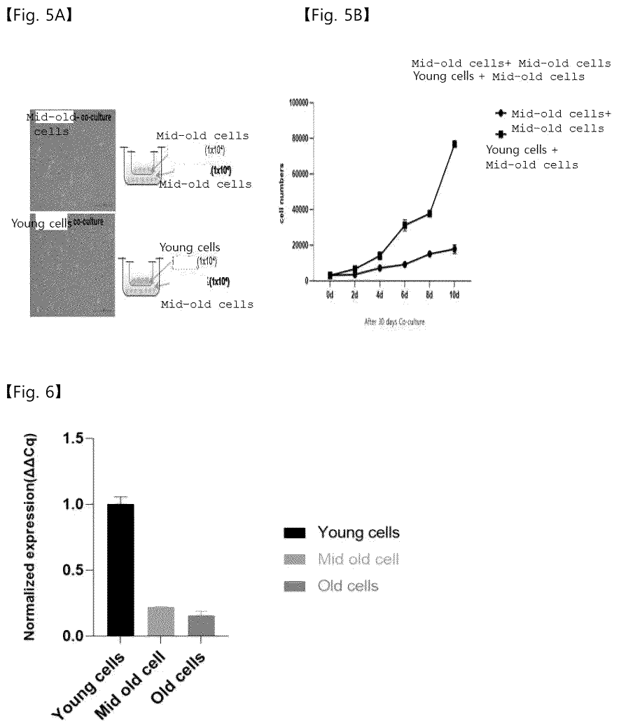

Then, the expression levels were compared between young cells, mid-old cells, and old cells using the expression level (value: 1) in young cells. As a result, both mid-old cells and old cells exhibited an Slit2 expression level reduced by more than 4 times compared to young cells (FIG. 6).

It was confirmed that young cells contain Slit2 at a higher concentration in compared to mid-old cells and old cells. The measured Slit2 concentration is shown in Table 5 below.

TABLE 5
Slit2 concentration

| Type of cells (3 × 10⁴) | Slit2 concentration (pg/ml) |
|---|---|
| Young cells | 76.92 |
| Mid-old cells | N.D |
| Old cells | N.D |

### Example 6: Confirmation of Induction of Reverse Aging in Mid-Old Cells by Slit2 Treatment

### 6.1 Confirmation of Morphology and Cell Count of Mid-Old Cells

To determine whether or not reverse aging in mid-old cells is induced by treatment with Slit2 protein secreted from young cells, mid-old cells were treated with recombinant human Slit2-N protein (purchased from PEPROTECH, catalog number 150-11, Source: HEK293 cells). Specifically, the mid-old cells were seeded and then treated with 100 ng/ml of recombinant human Slit2-N protein once every two days. Then, the morphology was observed on day 8.

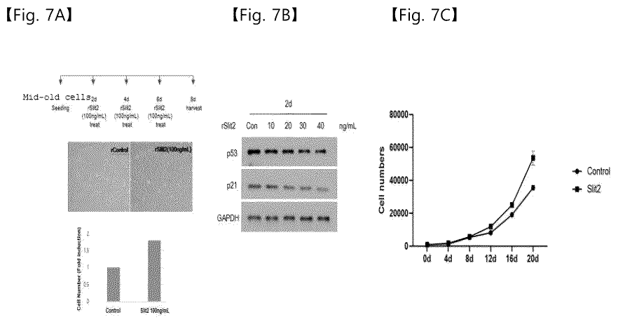

Compared to the control group, the number of cells increased, the overall cell size decreased, and cell proliferation was observed (FIG. 7A).

### 6.2 Confirmation of Expression of p53 and p21 in Mid-Old Cells

Mid-old cells were treated with recombinant human Slit2-N protein and then the expression patterns of p53 and p21 were observed. After treating mid-old cells with 10 ng/mL to 40 ng/mL of the recombinant human Slit2-N protein for 2 days, the protein was extracted from each cell using an extraction kit and the expression levels of p53 and p21 were measured using western blotting using antibodies against p53 and p21.

The antibody sequences used are shown in Table 6 below.

TABLE 6
Antibody sequences

| | | SEQ |
|---|---|---|
| Anti- | | ID |
| body | Sequence | NO: |
| p53 | MEEPQSDPSVEPPLSQETFSDLWKLLPENNVLS | 25 |
| | PLPSQAMDDLM | |
| p21 | MSEPAGDVRQNPCGSKACRRLFGPVDSEQLSRD | 26 |
| | CDALMAGCIQEARERWNFDFVTETPLEGDFAWE | |
| | RVRGLGLPKLYLPTGPRRGRDELGGGRRPGTSP | |
| | ALLQGTAEEDHVDLSLSCTLVPRSGEQAEGSPG | |
| | GPGDSQGRKRRQTSMTDFYHSKRRLIFSKRKP | |

The expression levels of p53 and p21 (CDKN1A), which are marker genes for aging, were compared between cells, and p53 and p21 (CDKN1A) gradually decreased when treated with recombinant human Slit2-N protein (FIG. 7B). This suggests that Slit2 protein is an effective substance that induces reverse aging of mid-old cells.

### 6.3 Comparison in Cell Count Between Mid-Old Cells and the Control Group

Mid-old cells were treated with recombinant human Slit2-N and a change in cell count was observed. Specifically, 3×10⁴ mid-old fibroblasts were seeded in a 12-well plate and 24 hours later, the fibroblasts were treated with 100 ng/ml of recombinant human Slit2-N daily for 20 days. Then, the cell count was measured every 4 days. The control group was treated with PBS. To measure the cell count, the adherent cells were converted into suspended cells using trypsin and then the cell count was measured with a microscope.

The result showed that, when the mid-old cells were treated with recombinant human Slit2-N, the cell count of mid-old cells significantly increased compared to the control group (FIG. 7C).

This suggests that cell proliferation was promoted when the mid-old cells were treated with recombinant human Slit2-N and that cell proliferation promotion is a characteristic of young cells and that treatment with recombinant human Slit2-N induces reverse aging.

### Example 7: Confirmation of Induction of Reverse Aging in Old Cells by Slit2 Treatment

To determine whether or not reverse aging is induced in old cells by treatment with Slit2 protein, the old cells were treated with recombinant human Slit2-N protein. Specifically, old cells were seeded and then treated with 100 ng/ml of recombinant human Slit2-N protein once every two days. Then, the morphology was observed on day 8.

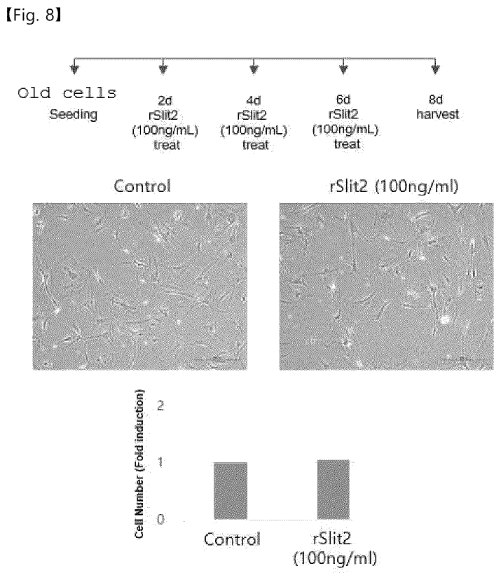

Unlike mid-old cells, it was found that there was no difference in cell morphology and cell count between old cells and the control group (FIG. 8).

This indicates that Slit2 protein secreted from young cells does not induce reverse aging in old cells.

### Example 8: Confirmation of Increased Activity in Aged Mice Upon Treatment with Slit2

To determine whether or not treatment with Slit2 protein secreted from young cells induces reverse aging in aged mice, aged mice were treated with recombinant mouse Slit2-N protein (SEQ ID NO: 27). Specifically, recombinant mouse Slit2-N protein was administered intraperitoneally to 22-month-old mice. Of each of the 9 male and 9 female mice aged 22 months, 4 mice were treated with PBS as a control group, and 5 mice were treated with 2 μg/mouse of recombinant mouse Slit2-N protein twice a week for a total of 3 weeks. One male mouse in the control group died during the experiment.

Then, after 3 weeks, the appearance of the mice was observed and the distance of activity of the mice was measured. The distance of activity of the mice was determined by measuring the movement path of the mouse in the cage for 1 minute by recording a video. The distance of activity of the mice of each mouse was measured three times and the mean thereof was expressed as the distance moved by the mouse.

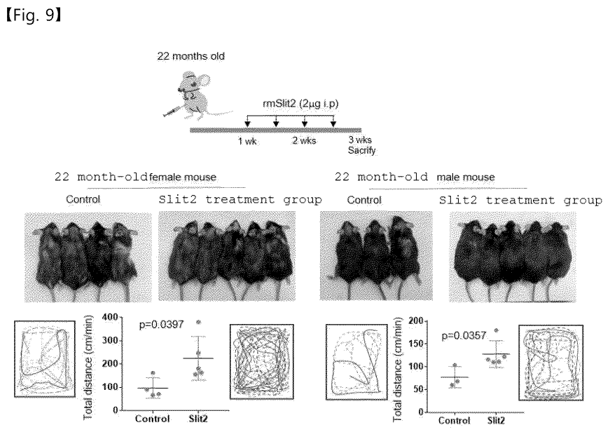

Compared to the control group, the activity distance of mice administered the recombinant mouse Slit2-N protein increased in both female and male mice and the result of appearance observation showed that hair loss was suppressed (FIG. 9).

This indicates that Slit2 protein secreted from young cells induces reverse aging.

In summarization of Examples above, when mid-old cells were treated with Slit2 protein, a substance secreted from young cells, the size of mid-old cells decreased comparable to young cells, the number of cells increased, and the increased expression of p53, p21, and inflammation-related genes which is a characteristic of the aging process decreased. This suggests that the aging is suppressed and reverse aging of cells that are undergoing aging is induced, and this indicates that the subject has a reverse-aging or anti-aging effect such as increased activity and suppressed hair loss.

### INDUSTRIAL APPLICABILITY

Slit2, a substance derived from young cells of the present invention, can improve the anti-aging induction effect while minimizing the side effects of conventional anti-aging methods, such as causing tumorigenesis and cancer progression, and thus is useful as a composition for inducing reverse-aging or anti-aging of senescent cells and for a method for inducing reverse-aging or anti-aging of senescent cells. In addition, Slit2 induces reverse-aging or anti-aging in the human body and thus is useful for the fields of healthy life, life extension, prevention or treatment of aging or aging-related diseases, health foods, or beauty.

Although specific configurations of the present invention have been described in detail, those skilled in the art will appreciate that this description is provided to set forth preferred embodiments for illustrative purposes and should not be construed as limiting the scope of the present invention. Therefore, the substantial scope of the present invention is defined by the accompanying claims and equivalents thereto.

### [Sequence Listing Free Text]

An electronic file is attached.

1. A method for reversing, delaying, and/or inhibiting aging process in a subject in need thereof, comprising administering to the subject an effective amount of a composition comprising Slit2 protein.

2. The method according to claim 1, wherein the Slit2 protein is a recombinant human Slit2-N(Slit2-N) comprising a sequence represented by SEQ ID NO: 1.

3. The method according to claim 1, wherein the reversing, delaying, and/or inhibiting aging process comprises reversing, delaying, and/or inhibiting aging process of senescent cells.

4. The method according to claim 3, wherein the senescent cells have one or more of the following characteristics:
(1) Decreased expression of Slit2 compared to young cells; and
(2) Increased expression of serum amyloid A1 (SAA1) compared to young cells.

5. The method according to claim 3, wherein the senescent cells are mid-old cells.

6. The method according to claim 5, wherein the mid-old cells have one or more of the following characteristics:
(1) Negative for SA-β-Gal staining; and
(2) Decreased p16^{INK4A} expression compared to old cells.

7. A method for preventing or treating a cellular aging-related disease in a subject in need thereof, comprising administering an effective amount of composition comprising a Slit2 protein to the subject.

8. The method according to claim 7, wherein the cellular aging-related disease comprises one or more selected from the group consisting of cancer, fibrosis, Alzheimer's diseases, arthritis, atherosclerosis and retinal degeneration.

9. The method according claim 7, wherein the composition is a pharmaceutical composition or a cosmetic composition.

10. The method according to claim 9, wherein the composition is the cosmetic composition having at least one function selected from the group consisting of skin whitening, skin wrinkle amelioration, hair color change, and hair loss relief or prevention.

11. A method for inducing reverse-aging or anti-aging of senescent cells comprising treating the senescent cells with Slit2 protein.

12. The method according to claim 11, wherein the senescent cells are mid-old cells.

13. The method according to claim 11, wherein the Slit2 protein is a recombinant human Slit2-N(Slit2-N) comprising a sequence represented by SEQ ID NO: 1.

14. The method according to claim 11, wherein the treating the senescent cells with the Slit2 protein is performed by co-culturing young cells expressing and/or secreting the Slit2 protein with old cells.

15. (canceled)
